(19) United States
(12) Patent Application Publication
(10) Pub. No.: US 2004/0250819 A1
(43) Pub. Date: Dec. 16, 2004

(54) Apparatus and method for detecting objects using tags and wideband detection device

(76) Inventors: William A. Blair, San Diego, CA (US); Jeffrey L. Port, New York, NY (US)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/810,623

(22) Filed: Mar. 29, 2004

Related U.S. Application Data
(60) Provisional application No. 60/458,222, filed on Mar. 27, 2003.

(51) Int. Cl. A61B 19/00

(52) U.S. Cl. 128/899

(57) ABSTRACT

An apparatus and method for the detection of objects in the work area such as surgical sites, including a detection tag affixed to objects such as used during surgery. The apparatus and method feature an interrogation and detection device with a transmitter for emitting pulsed wideband signals each including a signal prompting the tag element to provide a return signal, and a receiver for reception and analysis of the return signal from the tag element. Multiple pulse signals (of constant or varied height) emitted from the transmitter causes the return signals to build up in intensity at a detectable frequency above the ambient noise levels to facilitate detection of the tag element and object attached thereto. The device features an antenna portion containing a single or a plural ring-shaped antenna. Also, the pulsed wideband interrogation signal may be pulsed-width modulated or voltage-modulated, as two examples thereof.

This application claims benefit under 35 USC § 119(e) of U.S. Provisional Application 60/458,222, filed Mar. 27, 2003.

### INCORPORATION BY REFERENCE

This application incorporates by reference the entirety of U.S. Pat. No. 6,026,818, issued to the present inventors, entitled “Tag and Detection Device.”

### FIELD OF THE INVENTION

This invention relates to the detection of tagged objects and devices and, particularly, objects and devices utilized in body cavities during surgery.

### BACKGROUND OF THE INVENTION

During a surgical procedure, and especially in procedures where the chest or abdomen is open, foreign objects such as surgical sponges, needles and instruments are sometimes misplaced within the patients body cavity. In general any foreign object left within the body can cause complications, (i.e. infection, pain, mental stress), excepting objects such as clips and sutures that are purposely left as part of a surgical procedure.

Surgically acceptable procedures for detecting and removing foreign objects include counting the objects used in the operation. X-ray detection is also used to locate foreign objects. It is not uncommon, however, for object counts to be incorrect. Furthermore, even x-ray detection is not flawless. Despite the fact that objects such as surgical sponges, are typically embedded with an x-ray opaque material to make them more readily detectable, surgical sponges are often crushed into very small areas within a flesh cavity, whereby x-rays are not always able to sufficiently highlight them for detection. Furthermore, and most detrimentally, an x-ray is a time delayed detection method because of the requirement for film development (even with quick developing films). A patient will often be completely sutured closed before x-ray results are obtained, which may indicate the location of a foreign object within the patient. The detection delay may, therefore, result in the surgical team re-opening the patient, thereby increasing the morbidity of the operation.

Prior art techniques for the detection of foreign objects (aside from x-ray analysis) have typically either been prohibitively costly, involve detection devices which are too large to be meaningfully useful (i.e., they often impede utilization of the objects they are intended to locate), or simply do not provide effective detection. Exemplary techniques include marker or tag systems using radioactive, electromagnetic, magnetomechanical, or electromechanical detection techniques. A more detailed discussion of such prior known techniques are given in the background sections of the present inventors' above-named prior U.S. Pat. No. 6,026,818, which is hereby incorporated by reference in its entirety.

In theory, electronic locators should be suited to the detection of surgical sponges. As a practical matter, it is difficult to make a small tag element with sufficient signal strength for reliable detection at an economic cost. Increasing the size of a tag element may result in a detrimental effect on the utilization of the object it is intended to locate. For example, surgical sponges, a common item for which detection is desired, are useful because they can be deformed for use. However, deformation often distorts large tag elements and small tag elements may not provide sufficient signal strength for detection. A non-deformable large tag would effectively eliminate the usefulness of a sponge which is deformed for use. The inventors prior patent discusses this more extensively in connection with prior known schemes

Surgical objects such as sponges should be deformable to conform to body cavity work area. If the tags are shrunk and encapsulated so that they would take up a sufficiently small deformation resistant area within a sponge, they could be used without impeding the function of the sponge. However, as the area of the described tags is shrunk, their coupling will decrease, making them almost invisible to a typical detection system contemplated for use in surgery.

Therefore, there is a need for a cost effective tag element whose size does not cause a detrimental effect on the utilization of the object it is intended to locate and provides sufficient signal strength for detection.

### SUMMARY OF THE INVENTION

The present invention features a method and apparatus for detection of objects such as surgical sponges, which have remained in a patient after surgery. An apparatus of the invention comprises detection tags which are sufficiently small that they do not impede use of an object such a surgical sponge, or are larger but flexible, are reliable in discriminating detection, irrespective of the tags orientation, and are economical for widespread use in objects such as garments. The present invention also provides an apparatus and method/system for detecting tags that are sufficiently small for placement in a surgical apparatus.

Generally, the present invention comprises a method and apparatus for the detection of objects including foreign objects not intended to remain in a body cavity during or after surgery. The invention uses detection tags that do not adversely affect use of surgical apparatus and an interrogation and detection device providing reliable and strong detection signals.

I. An apparatus for the detection of an object contained in a work area includes a tag element affixed to a larger-sized object containing an electronic signal emitter within the protective means. The apparatus, which works well even with low Q tag elements, further includes an operable interrogation and detection member (or scanning/wand detection device), enabled to locate the tag element which is within a predetermined distance therefrom. The interrogation/detection member includes (i) first means for the emission of pulsed wideband signals in each coordinate direction, each wideband signal including a signal which prompts the tag element to provide a return signal, and (ii) second means for the reception and analysis of the return signal. The apparatus operates such that multiple pulsed signals emitted from the first means cause the return signals from the tag element to increase in the intensity at a detectable frequency sufficiently over ambient noise levels to facilitate detection of the tag element and object attached thereto. The interrogation and detection member contains an antenna portion shared for both transmit and receive functions and a handheld portion to which the antenna is detachably connected, the handheld portion contains the electronic transmitting/receiving components and the antenna portion includes a single or a plural ring-shaped antenna, the latter emitting a pulsed wideband signal as an electromagnetic signal in each coordinate direction of the multi-directional coordinate system employed.

II. A method for the detection of one or more objects in a work area such as in a surgical site including (i) attachably providing each foreign object with a much smaller low Q tag element which does not interfere with utilization of the foreign object, the tag element, which may be a low Q tag element, including means for responding to a wideband signal from a scanning detection device and returning a response signal centered about a specific but not a predetermined frequency. After completion of a surgical procedure, the method calls for scanning the surgical site with a scanning detection device containing a transmitter and receiver, the transmitter emitting either one of a pulse-width modulated wideband interrogation signal or a voltage-modulated wideband interrogation signal, the wideband interrogation signal containing a frequency at which the tag element responds with a signal response signal for each emitted pulse reaching the tag element, each pulse of the wideband interrogation signal being of such duration as to cause the return signals from the tag element to become cumulatively increased in intensity, resulting in a narrow band return signal having sufficient intensity to be distinguishable from background noise, to facilitate detection of the tag element and object attached thereto.

### BRIEF DESCRIPTION OF THE DRAWINGS

The foregoing and a better understanding of the present invention will become apparent from the following detailed description of the illustrated embodiments and the claims when read in connection with the accompanying drawings, all forming a part of the disclosure of this invention. While the foregoing and following written and illustrated disclosure focuses on disclosing example embodiments of the invention, it should be clearly understood that the same is by way of illustration and example only and is not limited thereto. The spirit and scope of the present invention are limited only by the terms of the appended claims.

The following represents brief descriptions of the drawings, wherein:

FIG. 1 is an exploded view of a bead type tag used in the present invention;

FIG. 2 is a cross section view of a thread type tag used in a second embodiment of the present invention;

FIG. 3A depicts a typical configuration for the hand held detector of the present invention;

FIG. 3B depicts a second configuration for the hand held detector of the present invention;

FIG. 4 depicts a thread for weaving into a surgical sponge having multiple beads of the type shown in FIG. 1;

FIG. 5 depicts a manner in which the tag bead of FIG. 1 is attached to a laparotomy sponge with a rivet attachment;

FIG. 6A is a block diagram of the electronics utilized in the detector of FIG. 3;

FIG. 6B is a block diagram of another embodiment of the electronics utilized in the detector of FIG. 3;

FIGS. 7A and 7B are graphical depictions of the wideband transmitter signal and the narrow return signal of the tag as differentiated from background noise, respectfully, and FIG. 7C is an explanatory illustration depicting transmitter energy decay between a wideband pulse excitation system and a traditional narrow band sinusoidal (high Q) excitation system and how they affect detection of the return signal from the tag.

FIG. 8 is a side view of another tag embodiment having three orthogonal tank windings to ensure detector wand cutting of a field line regardless of tag orientation and manner of wand deployment; and

FIGS. 9A and 9B depict detector wand deployment relative to a tag and various possible tag orientations with respect to marked field lines.

### DETAILED DESCRIPTION OF THE INVENTION

In one embodiment in accord with the present invention, a scanner comprises an electronic signal emitting detection device in the configuration of a movable wand with an interrogation ring (i.e., emitting antenna), and one or more tag elements (for each object to be detected) with each tag being of a size which is sufficiently small as not to impede the function of deformable objects such as surgical sponges to which the tags are affixed. In one embodiment, the tag is no greater than about 12 mm in its largest dimension. The tag element is electrically insulatively encapsulated in a bio-inert (if used in a surgical environment) hard plastic or glass in the form of a bead. Alternately, the tag is contained within a electrically insulative bio-inert flexible thread element (preferably elastic) which is attached to the object to be detected. In this embodiment the flexible, i.e., deformable, thread may be, for example, in about 3″ in length. If the object is a surgical sponge, the bead may be directly heat sealed to the threads of the sponge, adhered with a medically acceptable adhesive, or may be provided with an attaching thread for attachment thereto. Tags already in a thread configuration are directly woven into the sponge material where conditions of size are less stringent.

The tag comprises components that provide a return signal in response to the signal emitted from the detection device. The tags, generally, have a single signal emitting element such as an encapsulated miniature ferrite rod with wire winding, coupled with a capacitive means such as a capacitor for use in a bead embodiment, or a simple single loop wire, with winding, contained within an elastomeric coating as a thread element. An optional diode may be utilized to protect against tag-burn-out where an electric arc-weld type device known as a bovie, with applied electrical current, is used as a scalpel proximate thereto. Alternatively, the number of wire windings may be reduced to reduce any burn-out effect.

In one embodiment, the tag emits a small response signal, of general but not specifically known frequency, which would normally not be readily detectable because of its weak strength and non-predetermined nature. However, in accordance with the present invention, the signal emitting detection device in this embodiment utilizes pulsed signal emitting means with wideband capability, covering a signal range including that of the tag's response signal. The pulsed signals trigger a continuing response signal from the tag in its response frequency range which increases in intensity to the point where it becomes differentiable from background noise and is detected within the wideband range by the signal detector as an indication of the presence of the tag. Since the precise frequency of the signal response is not necessary or even pre-determined, expenses in electronics for emission and detection are typically reduced. The immediate turn off of the signal during pulses permits the quick location of the tag return signal (typically on the order of microseconds from transmitter turn-off), so that even a low Q tag may be utilized.

Tag characteristics in various embodiments, in addition to those mentioned above, and especially with respect to medical applications, include ruggedness, whereby the tag should be able to withstand high temperatures, and pressure from surgical instruments, (i.e. clamps) as well as all liquid immersions, bovie emissions, and high voltage cardiac defibrillation. The tags should be crush proof and the bead tags should be non-deformable under surgical use. Bio-inert plastics, such as are used with prosthetics, as well as bio-inert glass are useful for the encapsulation protection of the radiating elements.

The tags are detectable from a distance of at least about 12 to 18 inches from a handheld mobile detector. In addition, all orientations of the tag should be accommodated within the aforementioned range.

In one embodiment, the detector device is adapted to be hand-held and thus lightweight, and wand-like in configuration. This obviates the need for special bed installation or room adaptations, with detection equipment, as utilized in some prior art embodiments. The detector of the present invention is accordingly portable and movable with the patient.

In one embodiment, the detector device is provided with a radar-like system coupled with passive magnetic technology. The system for detecting surgical devices is defined by two basic elements:

a) a detection wand—the hand held unit which the surgeon or surgical staff member can use such as within approximately 12 to 18 inches of proximity to the patient and the surgical site, for verification of object removal; with the head of the device, being a single ring-shaped antenna or a plural ring-shaped antenna, which can be sterilized and replaceable, for open wound intrusion; with the wand containing transmission and receiving signal means; and

b) a tag which is excited by a signal from the detection wand, with detectable ring-back response.

In one embodiment of the present invention, the detection system uses a ring back technique (which excites a tag element, previously placed on a medical instrument or on/in a surgical sponge), by radiating magnetically coupled energy to the tag, through the loops of the antenna. The use of magnetic coupling is particularly preferred since magnetic coupling is almost inert to tissue and water immersion, conditions most prevalent in a surgical environment.

In operation, the transmit cycle from the detection wand utilizes a pulsed method to excite the tag(s) with signals over a fairly widebandwidth wherein sinusoidal components of the resonant tags exist in the pulses. This permits the use of economical tag components since center frequency is not required to be at close tolerances. Furthermore, the pulsed transmissions allow the system to pump more energy into the tag through several pulses, before ring back occurs, with extending ring back duration, thereby effectively increasing the range of detection to usable levels. The tag, when excited, transmits an image signal of its resonance decay, via magnetic coupling, back to the detection wand which contains a receiver circuit.

In one embodiment, the detection wand has a single loop antenna structure shared for both transmit and receive functions, with one loop for transmit and receive, through which the image of the numerous tag return signals (in response to the pulse signals) are processed, to create better signal to noise performance. The signal transmitter therein is multi-turn single loop, wherein band of operation frequency is lowered by reducing operational inductance of wire or by shrinking the diameter of the loop. Return signal averaging is used to “see” the signals (as a single enhanced signal) through noise. This “visibility” condition is a result of the multiple signals being added to each other directly whereas noise tends to add at its square root, thereby resulting in an enhanced single narrowband signal rising from the background noise levels. In one embodiment a custom box-car integrator is used for this purpose. In accordance with a further embodiment, the detection wand, according to the present invention, contains a multi-loop (multi-ring) antenna, for example, it may be structured with three (3) loop antennas arranged as three (3) mutually orthogonal rings, for both transmit and receive functions.

Signal processing technique according to the present invention allows the system to characterize a tag via its ring back response, and return (response) signals which are not indicative of tags are easily discarded by comparison to the characterized signature map. This provides detection results which are far superior to traditional ring-back systems which rely on either resonance or center frequency detection based on phase shift around a center frequency.

Traditional RFID, on the other hand, is based on a electromagnetic coupling of specific frequency signal between a transmitter-receiver and tags operating at a resonant frequency. During transmit cycles the tags are excited, and then ring back a return signal post transmission. Typically, the transmitter is tuned tightly (e.g. within 3%) of the center frequency and is designed to have a high “Q” or power transfer. Likewise, tags are also designed to have the highest Q and tightest control of center frequency possible. This approach is, unfortunately, limiting in range, as the tag coupling area (size) is substantially reduced. In practice, detection of 2 mm sized rod tags, typically, are limited to 6″ or less operating ranges with most systems. The problem is the tag response energy (for tags with small area of coupling) is often time undetectable in the midst of the ambient background noise of the transmitter energy decay cycle. Reliance on resonance characterization typically requires either a very tight tolerance of tag components, and or a sweep transmitter function. In contrast, the present system may be wideband for transmission, but extremely discriminating between tags and other objects, without added complexity.

“Q”, which refers to the quality factor, relates to, for example, the energy transfer between tag and detector in an RFID electromagnetic system. Function is not dependent alone on the Q of the tag but on the energy transfer between the tag and detector at a given distance and coupling (or at an energy level and effective area of cross section between a tag and detector). The present invention will function well under lower Q operation characteristics than that typified by a narrow band (traditional RFID) approach, because the method of excitation and ring back are managed differently. The apparatus and method/system of the present invention features an interrogation/detection scheme, which can use wideband methodologies.

In electronic detail of one embodiment, the pulse generator is set to emit pulses to excite a range of frequency components. The pulses are controlled for duration and interval to maximize energy transfer to the tag over a desired bandwidth. The pulses are sent through a driver and amplified to an appropriate signal level and a transmit amplifier is designed to shut off quickly. To accomplish this, an untuned transmitter is utilized, which relies on the pulse method to insure energy transfer to the resonant tag.

It is noted that tuned transmitters have been used to excite resonant tags because the energy transfer efficiency to the tag is high. However, in accordance with the present invention, an untuned transmitter is used because of its useful shut off time with respect to pulsed signals. The use of pulses, as described, makes up for the poor energy transfer since multiple pulses build additive energy into the tag.

In accordance with the present invention, the receiver is also wideband whereby it can see tags over a wide spectrum benefiting from fast transmitter signal decay. The receiver further comprises limiters to insure that the transmit cycle does not saturate it. Once a signal is amplified by the receiver, it is sampled by a sample-and-hold circuit/analog to digital converter, or, in one embodiment, a digitally controlled phase sensitive averager.

Use of an analog to digital converter is useful when an optimal DSP (Digital Signal Processing) technique is to be applied. The time when a signal is sampled is controlled from a TX inhibit clock and control logic in order to insure that a signal captured is at the appropriate time from the transmitter shut off time. Signal processing such as averaging is applied either in through clocking with the sample and hold or summing circuit or by a microprocessor (μP) or a DSP. In effect, the averaging technique is similar to a synchronous detector creating a super narrow digital like band pass function (increasing signal to noise of tag return signal).

If a microprocessor is used, it can then store the output of an analog to digital converter. In addition, the microprocessor may be used to characterize ring signature. Depending on the level of complexity of signal processing which may be necessary, the DSP may not be needed or may be external.

In normal operation the transmitter, while it is exciting the tag, is blocking any possible return signal from the tag. That is the system is, in effect, half duplex. Accordingly, one problem encountered in design of a detection wand is reducing the turn off time of the transmitter signal. When the transmitter is on, the tag will be excited and will also radiate a return signal. At the time the transmitter is turned off, the tag is at its peak amplitude of a radiated signal. But the transient transmitter signal is still present, so the tag signal will not be easily visible until some time later. The transient signal from the transmitter is from capacitive and inductive components in the transmitter/receiver circuitry and exists even if the transmitter is shunted at turn off. Accordingly, the transmitter/receiver circuit of the present invention is made wideband using low loop capacitance in the analog front end. Thus, greatly improved distance or sensitivity are achievable with small low cost tags having weak signal return.

In one embodiment, the present invention uses the same excitation frequency component to power the tag and to receive ring back from the tag while being wideband in design, and the combination of wideband design coupled with signal processing technique allows for enhanced performance of range, and reduced tag cost and tag size.

Since surgical procedures involve traumatic procedures, the present invention permits the tags to remain attached to the sponge or instrument to be located. Accordingly, the present invention encompasses many ways for the detachable resistant attachment of the tag to the sponge or instrument. Because of the more severe use (i.e., deformation), involved in sponge use, good attachment thereto is most problematic.

The tags of the present invention in one embodiment are made into a hard object to resist deformation, and each is coated with an insulative, bio-friendly and inert shell and, according to one example of a preferred embodiments, is made of a ferrite core with some loop wire and a capacitor (optionally with a diode for burn-out protection) contained, for example, within a 5 mm-12 mm oval shape plastic shell, although not limited thereto. A string is integrated into the encapsulating shell to accommodate the tag to be integrated in the cloth manufacturing procedure used in manufacturing the sponge.

In another embodiment a rivet button cell is attached to a corner of the sponge material. For a lap pad, the button cell may be added to the lap pad drape loop extension. Alternatively, the tags can be adhered to the substrate to be detected by materials such as surgical adhesive implantable FDA USP class VI.

As described above, the detector and tag provide positive signal evidence (audio or visual, e.g., at sufficient signal strength, an LED signal lights up) that a sponge or similar object remains in a body cavity (or that an object is located within a scanable area). However, because of electronics and cost considerations, exact location is not as desirable and is often not as necessary, since mere knowledge of the presence of a sponge is sufficient for a surgical team to quickly manually locate the object within a body cavity (i.e., in the location where sponges were actually used). It may however, be desirable to actually locate the object or at least narrow the range of the site in which the object may be located. Accordingly, detectors of different ranges may be utilized, once the presence of an object has been determined. These detectors may be modified in antenna (or loop) dimension or in power used for signal emissions. With regard to the latter, a single detector can be used, e.g., one with variable power output (the level of which is controlled in stages with successively narrowed ranges) to locate the “lost” object with greater precision.

A fixed repetition rate of putting out pulse sequences to excite tags may be susceptible to continuous wave noise (i.e., signals close to the tag frequency). Accordingly, in a further embodiment, the pulse signal frequency is varied in random fashion to make it very difficult for continuous wave noise to affect the system, since it will become very out of phase with the tag resonance. This is similar to a spread spectrum approach in frequency hopping.

In a further embodiment, broad band noise such as is generated with lightning or use of a bovie, is not treated as an adding sample of pulse signal return by tag and is excluded by sampling and shunting to ground.

To ensure that null field couplings are not encountered by the detector wand, such as may result from orientation of the tags, the tags may be initially configured with three orthogonal tank windings. These comprise horizontal and vertical tank circuits in addition to the center tank circuits described for use with the tags. With these additional tank circuits, there is always a loop coil on the tag which will have field lines from the detector wand cutting or passing therethrough. This avoids any misreadings caused by simple placement of the detector wand on the site, instead of with a lateral waving motion.

It is understood that with the three orthogonal tank windings, the geometry of the tag may be varied, such as to enhance its reproducibility, and the various tag shapes may include cubic, cylindrical, spherical, etc.

### DETAILED DESCRIPTION OF THE PREFERRED EMBODIMENTS

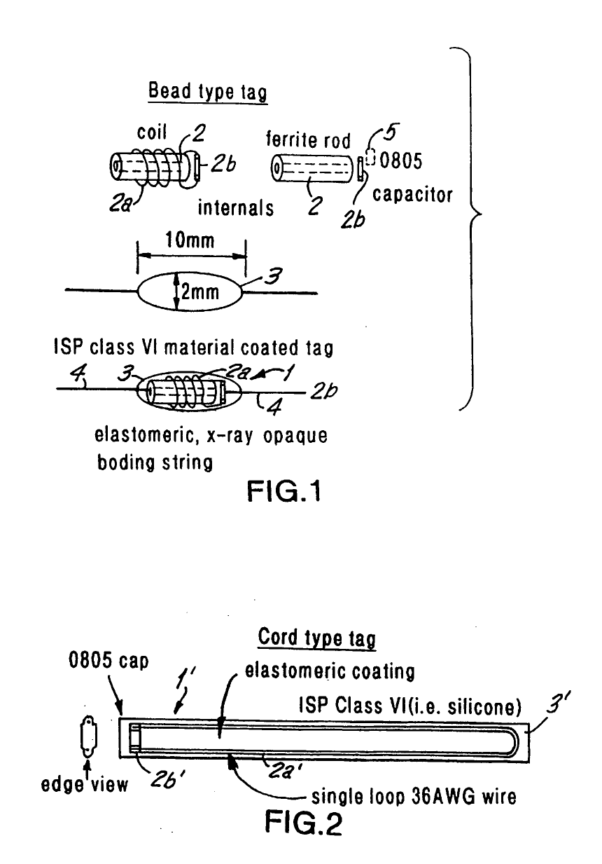

With specific reference to the drawings, in FIG. 1, an example first embodiment of a tag (tag element) 1 in the form of an oval bead, with dimensions of about 10 mm length and 2 mm diameter, although not limited thereto, is shown with encapsulated miniature ferrite rod 2 with coil 2a and capacitor 2b. A protective shell such as the hard bio-inert plastic 3 encapsulates the coil, rod and capacitor to protect it from damage from pressure and body fluids. Elastomeric, x-ray opaque bonding strings 4 serve as a means by which the bead is attached to a surgical sponge. Because of its very small dimensions, the bead does not impede deformation of the sponge to which it is attached. Diode 5, shown in dotted line outline, is optionally included as electrical protection against electrical burnout from proximate electronic instruments. In one embodiment, the time constant of the tag may be selected to avoid introducing an unacceptable delay between the transmission of the interrogation signal and the return signal from the tag.

In FIG. 2, an example second embodiment shows a tag 1′ in the form of a thread with single loop wire 2a′ (shown as an elongated U-shaped wire) with capacitor 2b′ enclosed within an elastomeric coating or sleeve 3′. The diameter of such tag is similar to that of the bead embodiment of about 2 mm but because of its high flexibility, its length can be greater, e.g., including to about 3″ (about 75 mm) and it provides its own thread-like attachment means.

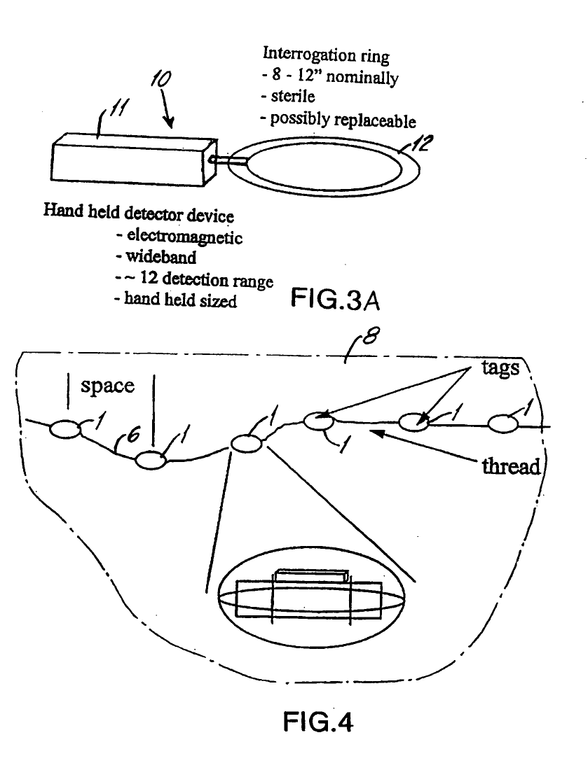

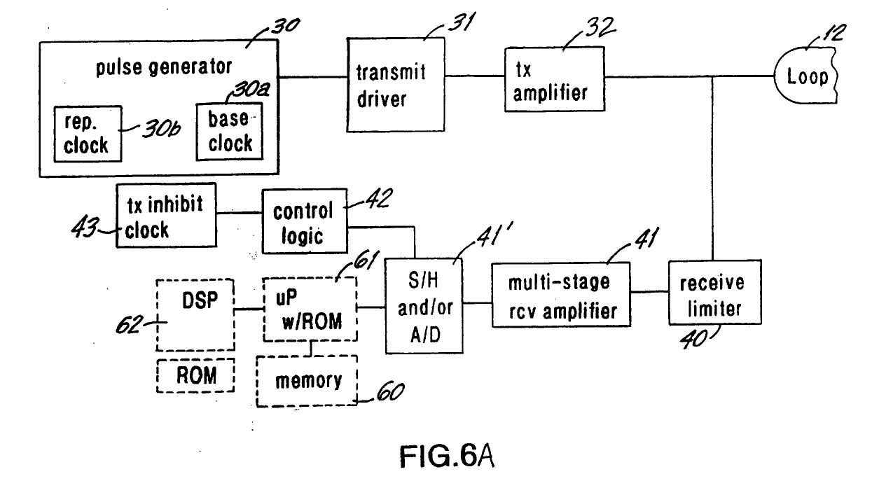

The bead 1 tag element of FIG. 1 and the thread (or cord type tag element) 1′ of FIG. 2 are detected by a wand (scanning) detection device such as interrogation/detection device 10, shown in FIG. 3A, which comprises handle 11 (with contained electronics, e.g., as schematically set forth in the block diagram of FIG. 6A or FIG. 6A with 6B, including, with power supply source of battery or transformer and standard AC electrical connection), although not limited thereto. Interrogation and receiving ring 12 of the device 10 is an antenna ring with a nominal diameter of between 8-12″, although not limited thereto. Since the ring 12 is in proximity to the surgical site for detection, it is sterile and removable for sterilization or replacement. It may also be replaced with, for example, a smaller diameter ring for more precise location once it has been determined that a “lost” object is present.

As mentioned above, the interrogation and receiving ring 12, shown as a multi-turn single loop antenna, although not limited thereto, functions to transmit an electromagnetic signal to the tag 11 and to receive a response signal from the tag 1. The size and geometry of the ring 12 are, in practice, chosen such that the ring 12 is capable of receiving the response signal from the tag 1. For instance, where the ring 12 is too small, the ring 12 may, upon a sweep of a patient's body, miss detecting the tag 1. Reducing the size of the ring 12 beyond a certain point also limits the distance from the patient's body at which the interrogation/detection device 10 can detect the tag 1. In practice, as one example, using a ring 12 with a 6″ diameter, in comparison to a ring 12 with the aforementioned 8-12″ diameter, does not affect the distance from the patient's body at which the ring 12 can detect the tag 1. In one example embodiment, the ring 12 may be a solenoidal coil.

Referring to FIG. 3B, in an example alternative embodiment of an interrogation and detection device (or wand detection device or scanning detection device) of an apparatus and method therefor for detecting an object, to improve the ability of the device 10 to detect the response signal from the tag 1, the device 10 includes three rings 13, 14, and 15. The three rings are, in this example embodiment, mutually orthogonal to one another. Rings 13, 14, and 15 transmit the electromagnetic signal to the tag 1 in the x, y, and z-directions, respectively. The three orthogonal rings may be embedded (contained) in a housing. This embodiment is useful in that a tag, oriented to receive an electromagnetic signal in only one of the x, y, or z-directions, is assured of receiving the emitted signal and, as such, transmitting a response signal (i.e., a return signal) to the device 10. Two modes of operation are possible, simultaneous or round-robin transmit excitation. The round robin mode provides best receiver sensitivity given directionality of energy over time.

In the round robin scheme, the three rings 13, 14, and 15 transmit in succession, such that only one of the three rings 13, 14, and 15 is transmitting at any one time. Following a transmission by one of the rings 13, 14, and 15, there is a brief intermittent period before transmission by another of the rings 13, 14, and 15 is effected. During this intermittent period, the rings 13, 14, and 15 are used to determine whether a response signal is forthcoming from the tag 1. The intermittent period is, however, on a very small order of time, such that the tag 1, even where it changes its orientation, will receive the electromagnetic signal and, consequently, send a response signal to the device 10, allowing the tag 1 to be detected.

The round-robin method requires that the clocking between transmit cycles and the inhibit cycles of each ring be coordinated properly to avoid overlap to the prior excitation and receive period of the last used ring. It should be noted that the three orthogonal rings provide receiver signal information in three planes. As the goal of the device is to detect the presence of tags, the receiver information is aggregated into a threshold measurement for tag(s) presence. As a modification thereof, if desired, the receiver circuitry could have three receivers and provide signal information in three planes creating a digitization or special location information.

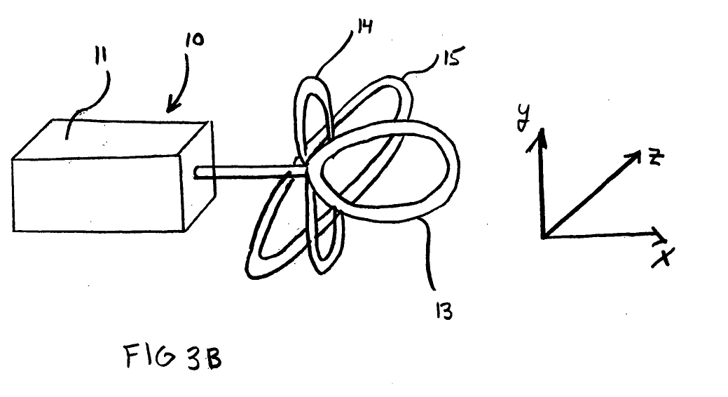

As shown in the example illustration in FIG. 4 of affixing multiple tags (tag elements) to an object such as a surgical sponge, a thread 6 with multiple beads 1 may be bonded, stitched or woven into the fabric of surgical sponge 8, as a low cost detection tag with increased signal strength.

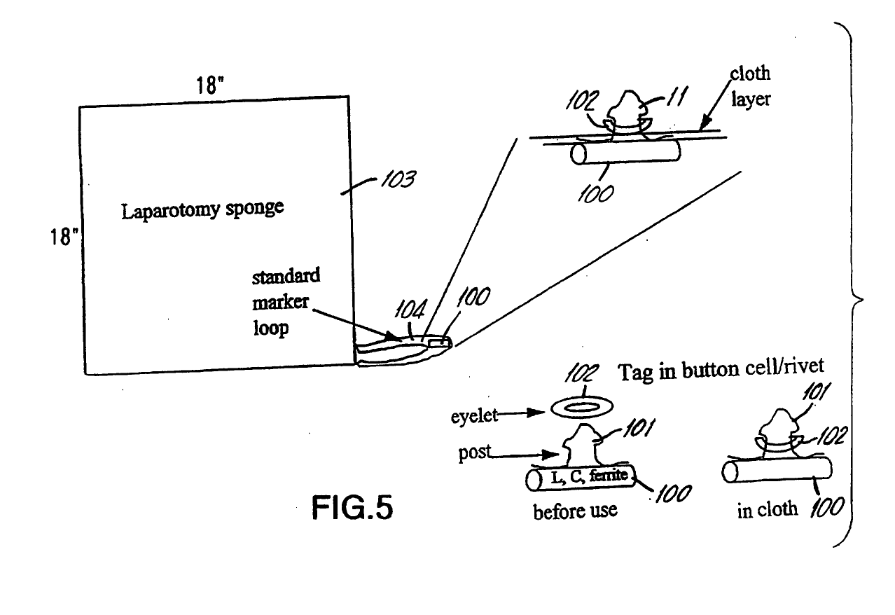

In another embodiment, as shown in FIG. 5, tag 100 is provided with a rivet attachment member 101 and eyelet 102 whereby the tag is attached to a laparotomy sponge 103 which is typically provided with standard marker loops 104.

In the two example embodiments shown in FIGS. 3A and 3B, although not limited thereto, the electronics for both interrogation and reception of return signal are contained within the device 10. According to an example showing thereof in FIG. 6A, for the interrogation operation, the device 10 contains a pulse generator 30 with a base clock 30a and rep. clock 30b, as well as transmitting driver 31 and tx (transmitter) amplifier 32 as the signal goes to antenna loop or ring 12. For receipt and analysis of a return signal, the loop ring 12 receives the signals from any of the tags which may be present within the interrogation range thereof (e.g, of up to 18″ or more and, generally, within the range of 12-18″), with the signal passing receive limiter 40, multi-stage receiving amplifier 41, and sample-and-hold (S/H) and/or A/D converter 41, which is controlled by control logic 42 and tx inhibit clock 43. Random access memory (RAM) 60, and μP (microprocessor) with ROM (read only memory) element 61 and DSP (digital signal processor) with ROM 62, shown in dotted lines, control and analyze the pulse signal and return signals received by the S/H and/or A/D 41.

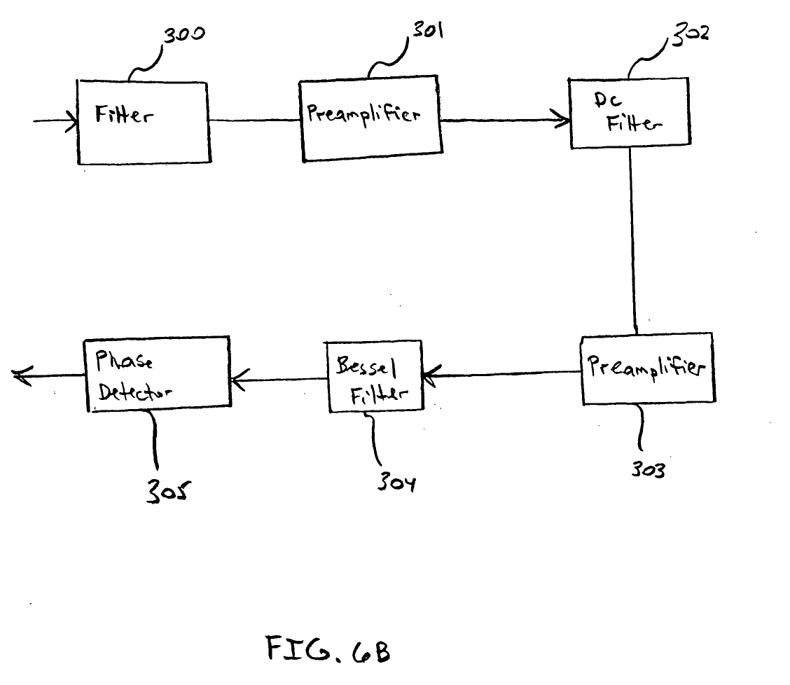

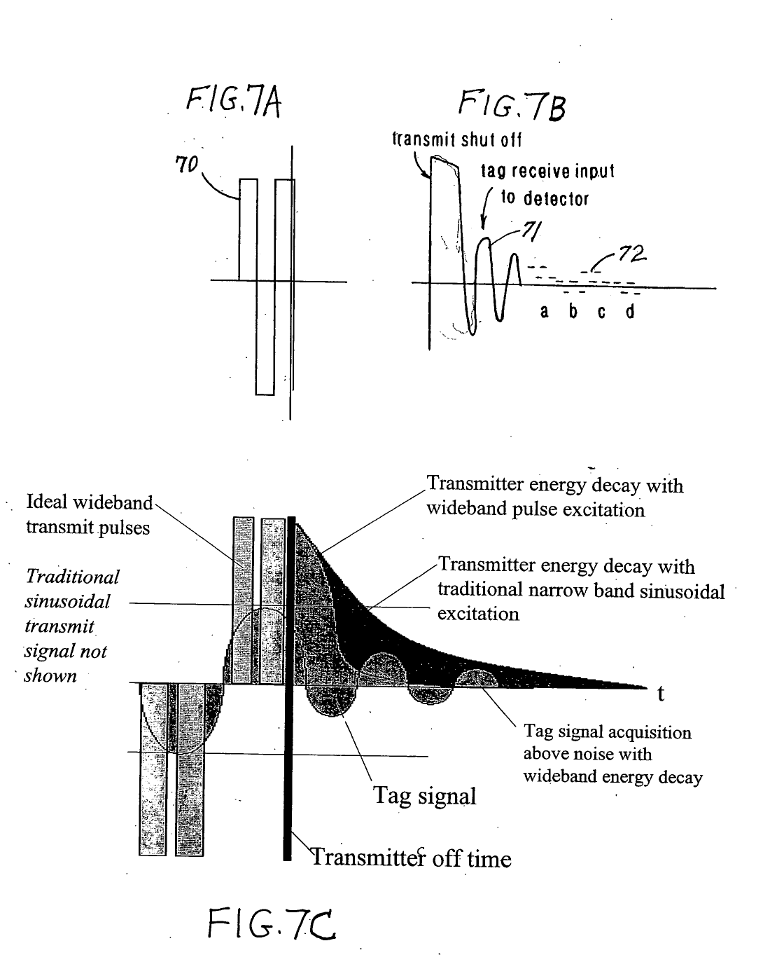

The return signal 71, shown in FIG. 7B, which commences with the shut off of the transmit signal 70 (see FIG. 7A), rises in intensity (e.g., magnitude) to a level easily distinguishable from the background noise 72 as the detector nears the tag thereby facilitating detection of the location of the surgical sponge (or other object) attached to the tag, for example, by triggering an audio and/or visual alarm. This is because the tag element is excited using a low Q wideband transmitter. Although this may be considered a less efficient forward energy transfer approach, it does have the ability to clamp the transfer decay rapidly and allow the tag signal to be seen earlier in its return decay. In effect, this works because the tag signal can be seen (processed) out of the noise floor, which is reduced by application of a wideband transmitter/receiver device. An explanatory illustration of this including a comparison showing between that of a wideband decay and narrow band high Q excitation is shown in FIG. 7C.

Incidentally, a digital μP embodiment may be used to implement most of the component parts of the above implementation in software and mixed signal chip technology. It is well known (digital signal processing) to a skilled digital design engineer to apply, for example, a single chip μP or DSP (digital signal processing) parts to handle clocking, filtering, digital amplification, threshold detection, phase detection, digital pulse generation and many other signal-processing functions to make the aforementioned transmit and receive system. Also, numerous combinations of known filtering schemes can be applied to achieve improved signal discrimination. In general the application of digital signal processing techniques with RFID (Radio Frequency Identification) have in the past been less important because the transmit/receive systems employed narrow bandwidth operation schemes using highly tuned, high Q embodiments. The use of digital signaling techniques with a wideband approach, the present inventors have found, enables attainment of a characteristically different level of performance in signal discrimination, and, in particular, enables the ability to discriminate tags over a wide frequency range with software (or firmware) control and no physical redesign. The implementation of additional filtering can be applied through software such as in connection with Bessel filtering, which effectively narrows the noise bandwidth and thereby enhances the detection range of the receiver portion of the scanning detection device.

Turning back to the electronic block diagram in FIG. 6A, to improve the range of the device 10, several desirable adjustments are made to the components therein. For instance, it is desirable to have a high drive voltage, as doubling the drive voltage results in about a 10% increase in detection range (i.e. distance) of the device 10. The drive voltage should, however, because of voltage ratings of the capacitor 2b used in the tag 1, be kept within certain limits. Typically, the drive voltage of the pulsed waveform should be kept within +/−35 volts.

Further, to prevent the device 10 from detecting low-frequency noise induced in a metallic object located in proximity to the device 10, such as, for example, a proximate metal table, the pulse generator 30 is chosen to generate a series of pulses centered about 0V. In other words, the pulse generator 30 generates a series of pulses having a DC value of 0V. In such a manner, equal amounts of positive and negative energy are transferred to a proximate metal object. As such, the eddy currents created in the proximate metal object are minimized or eliminated, such that the device 10 is prevented from coupling to loop transients in the proximate metal object. The pulse generator 30 may be implemented to generate such pulses using, for example, a balanced bipolar driver circuit.

To further increase the signal to noise ratio, the pulse generator 30 is implemented in such a fashion as to modulate the transmitted pulses. In one embodiment, for example, multiple drive voltage levels are used. The voltage levels of the pulses are varied over time. For this embodiment the transmit driver 31 in FIG. 6A is controlled by the pulse generator (likely implemented as software/firmware routine in μP or DSP), and can switch or modulate a variety of drive voltages. Not shown are the voltage sources, and modulation elements, as these should be well established for one familiar in circuit design art.

In another embodiment, pulse width modulation (PWM) and repetition management are first employed to shape the drive waveform. Effectively, in such implementation of an interrogation and detection device, the pulse generator 30 in FIG. 6A, which, for example, may be implemented using firmware in μP or DSP, alters pulse width and number of digital signals applied to the transmit driver by turning them on or off creating pulse management. Modulation either via multiple drive voltages or pulse width variation can also help to discriminate tag signals from noise sources, as a response to modulation by the tags will produce different spectral behavior than other noise sources. This is especially valuable in an implementation with a DSP where signal processing using, for example, FFT (Fast Fourier Transforms) might be used to validate a tags signature in the frequency domain. Signal processing techniques such as using FFT are well established with regard to radar systems and should be available to one familiar with DSP based digital design technology.

To further increase the signal to noise ratio, the pulse generator 30 is, in another embodiment, implemented as a spread spectrum driver. That is, the repetition rates of the transmit drive pulses are varied in a more random manner over time or are made less periodic. Such implementation can be effected through using DSP to create random synchronous excitation, a technique well described to those familiar with signal processing in wideband systems. By implementing the pulse generator 30 as such, the device 10 is made much less sensitive to constant frequency sources that may be within approximately 5% of the resonant frequency of the tag 1. To implement the pulse generator 30 as such, the time interval between successive drive pulses is altered. Since the phase detection, as discussed below, is slaved to the drive timing, operation of the device 10 will not be adversely affected. However, the relative phase of external signals will change from pulse to pulse and will thus be averaged out in the phase detector. In one such embodiment, the drive timing is changed by one-half cycle of the tag frequency with each drive pulse. The resulting spectrum is complex enough that it is not likely to be matched by other kinds of transmissions in this band.

Traditional RFID, on the other hand, is based on a electromagnetic coupling of specific frequency signal between a transmitter-receiver and tags operating at a resonant frequency. During transmit cycles the tags are excited, and then ring back a return signal post transmission. Typically, the transmitter is tuned tightly (e.g. within 3%) of the center frequency and is designed to have a high “Q” or power transfer. Likewise, tags are also designed to have the highest Q and tightest control of center frequency possible. This approach is unfortunately, limiting in range, as the tag coupling area (size) is substantially reduced. In practice, detection of 2 mm sized rod tags, typically, are limited to 6″ or less operating ranges with most systems. The problem is the tag response energy (for tags with small area of coupling) is often time undetectable in the midst of the ambient background noise of the transmitter energy decay cycle.

Further, the receive limiter 40 and multi-stage receiving amplifier 41 may, alternatively, be implemented as illustrated in FIG. 6B. As shown, the received signal from the tag 1 may be passed through an initial filter 300, preamplifier 301, a DC Filter 302, and a second preamplifier 303. Additionally, a critically damped second or third order filter, such as, for example, a Bessel low pass filter 304 may be provided, also, at the input to a phase detector 305 along with detector 305. By using the Bessel low pass filter 304 to reduce the noise bandwidth, a 5% increase in detection range of the device 10 is achieved.

Implementation of the functional blocks shown in FIG. 6B was built largely with discrete components. It should also be noted that the use of two discrete pre-amplification stages may not be necessary in a DSP based implementation. After DC filtering such as by 302 or anti-aliasing, the signal may be completely processed by a DSP. Commonly, mixed signal DSP chips offered for generic applications can be employed to model all or part of functional components 303-305, making the components stages programmable. It is also understood that one could add many filters and gain (amplify) stages digitally. For implementations at higher frequencies, where effects from noise sources over the wider frequency range can be more pronounced, additional such filtering and signal gain techniques would be desirable. DSP art is well known and supported by a host of vendors. One familiar with the art should have no trouble implementing these component parts or selecting proper filter libraries to achieve an acceptable DSP operation with regard to realizing the various example embodiments disclosed in this specification of the present invention.

The phase detector 305 may be implemented in a variety of manners. In one embodiment, the phase detector 305 uses the transmitted pulsed waveform to locate the response signal received from the tag 1. For example, when the pulse generator 30 generates a pulse, the phase detector is turned off. When, on the other hand, the pulse generator 30 is not outputting a pulse, the phase detector is turned on. In such a fashion, the phase detector is better equipped to locate the response signal from the tag 1. In another embodiment, the phase detector 305 could be modified to specifically reject the recovery tail from the transmitter, although this may not be necessary since the shaping of the pulsed waveform, as described above, will tend to accomplish the same thing.

In the current implementation, the output of the phase detector is integrated via S/H and/or A/D converter 41′ to produce a threshold signal for triggering the alarm, indicating the presence of a tag or tags. In another embodiment using DSP methods for signal processing, like FFT and other transforms, the signal is compared for width, pulse shape, or match to a much more articulate set of image criteria (not just integration level), thus improving the rejection of false signals and providing more sensitivity potential to alarm outputs. One familiar with the art should have no trouble implementing the component parts in the example embodiments shown in FIG. 6A and FIG. 6A combined with FIG. 6B, or the various other embodiments and modifications disclosed herein, including additional obvious modifications thereof.

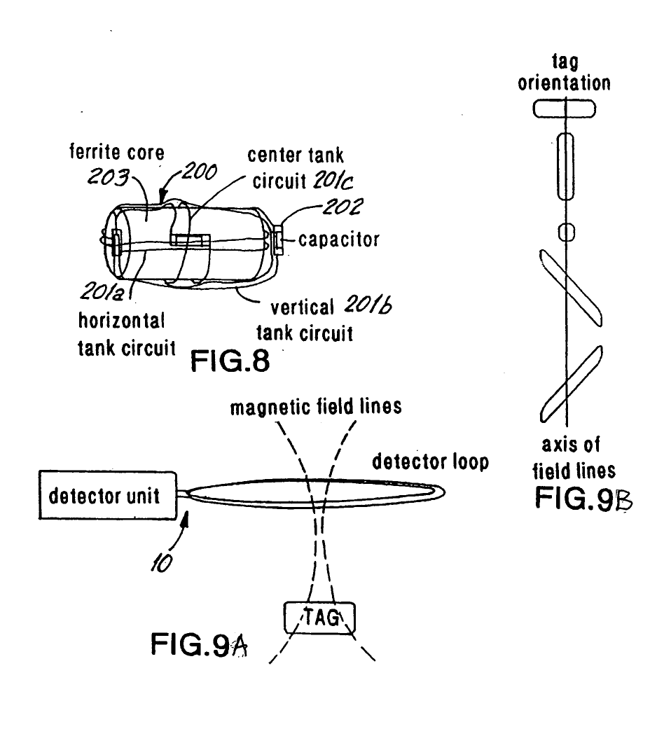

As shown in FIG. 8, a tag 200 which is detectable regardless of orientation is provided with three orthogonal tank circuits 201a, 201b and 201c, respectively shown as horizontal and vertical in addition to the center tank circuit previously described. A single capacitance element 202 is utilized in addition to the ferrite core 203. As shown in FIGS. 9a and 9b, deployment of detection wand 10 detects magnetic field lines regardless of tag orientation.

This concludes the description of the example embodiments. Although the present invention has been described with references to a number of illustrative embodiments thereof, it should be understood that numerous other modifications and embodiments can be devised by those skilled in the art that will fall within the spirit and scope of the principles of this invention. More particularly, reasonable variations and modifications are possible in the component parts and/or arrangements of the subject interrogation/detection device and tag elements and method thereof of employing the same in the detection of object(s) within the scope of the foregoing disclosure, the drawings and the appended claims without departing from the spirit of the invention. In addition to variations and modifications in the component parts and/or arrangements, alternative uses will also be apparent to those skilled in the art.

What is claimed is:

1. An apparatus for the detection of an object contained in a work area, comprising:
a tag element affixed to a larger-sized said object, said tag element comprising an electronic signal emitter contained within a protective means; and
an electronically operable interrogation and detection member, enabled to locate the tag element within a predetermined distance therefrom, comprising (i) first means for the emission of pulsed wideband signals in each coordinate direction of a multi-directional coordinate system, each wideband signal including a signal which prompts the tag element to provide a return signal, and (ii) second means for the reception and analysis of the return signal,
wherein multiple pulsed signals emitted from the first means cause the return signals of the tag element to increase in intensity at a detectable frequency sufficiently over ambient noise levels to facilitate detection of the tag element and object attached thereto.

2. The apparatus according to claim 1, wherein the tag element is bead shaped and includes a response signal emitter encapsulated in a bio-inert hard shell, the response signal emitter comprises a ferrite rod and a coil wire therearound with a means for providing capacitance coupled thereto.

3. The apparatus according to claim 2, wherein the bead shaped tag element further includes means for attachment to the object.

4. The apparatus according to claim 3, wherein the work area is a surgical site, the object includes a surgical sponge and said means for attachment comprises flexible threads anchored in the bead to enable attachment to threads of the sponge.

5. The apparatus according to claim 3, wherein the work area is a surgical site, the object is a laparotomy sponge and said means for attachment comprises a rivet attachment member and an eyelet member in which the tag element is attached to a marker loop of the laparotomy sponge.

6. The apparatus according to claim 2, wherein the response signal emitter further comprises a protective diode coupled to prevent accidental burn-out thereof caused by proximate electrical equipment.

7. The apparatus according to claim 1, wherein the electronic signal emitter comprises a single loop, with winding, contained in an elastomeric coating as a thread element.

8. The apparatus according to claim 1, wherein the electronic signal emitter comprises a single elongated U-shaped wire with a capacitance element coupling the ends thereof, and enclosed within a flexible casing.

9. The apparatus according to claim 1, wherein the interrogation and detection member is comprised of an antenna portion shared for both transmit and receive functions and a handle portion to which the antenna is detachably connected, the handle portion contains first electronic means for pulsed wideband transmission and second electronic means for detecting and analyzing the response signals, and the antenna portion includes plural ring-shaped antennas for the emitting of a pulsed wideband signal as an electromagnetic signal in each coordinate direction of the multi-directional coordinate system employed.

10. The apparatus according to claim 9, wherein the antenna portion includes three mutually orthogonal ring-shaped antennas for the transmitting of the electromagnetic signal in the X, Y and Z-directions, respectively, of an X, Y and Z-coordinate system.

11. The apparatus according to claim 1, wherein the work area is a surgical site and the tag element is of such relatively small size as not to impede the functional use of an object to which it is affixed, the object being either deformable or non-deformable.

12. The apparatus according to claim 1, wherein the first means comprises an untuned transmitter.

13. The apparatus according to claim 1, wherein the first means includes an electronic portion configured to produce pulse-width modulated (PWM) wideband signals.

14. The apparatus according to claim 1, wherein the first means includes an electronic portion configured to produce voltage-modulated wideband signals.

15. The apparatus according to claim 1, wherein the first means includes an electronic portion configured to produce pulsed wideband signals in which one of either the voltage levels of pulses are varied over time or pulse width variation is effected over time to enhance discrimination of the tag element response signals from ambient noise.

16. The apparatus according to claim 1,
wherein the first means includes an electronic portion configured to produce either one of pulse-width modulated wideband interrogation signals or voltage-modulated wideband interrogation signals, and
wherein the second means includes (i) a second electronic portion configured to receive and analyze the narrowband return signals, the second electronic portion including a wideband receiver containing filter and pre-amplifier circuits to reduce noise bandwidth of incoming signals and increase detection range of the interrogation and detection member, and (ii) a signal processor to transform the response signals into a resulting narrowband return signal having sufficient strength to be distinguishable from ambient noise.

17. The apparatus according to claim 1, wherein the tag element is a low Q tag element.

18. An apparatus for electronic detection of an object contained in a work area comprising:
a first electronic circuit, coupled to a transmit/receive antenna, configured to emit either one of pulse-width modulated wideband interrogation signals or voltage-modulated interrogation signals,
a tag element affixed to a larger-sized said object, the tag element being adapted to respond to each pulse of an interrogation signal with a relatively small narrow return signal centered about a specific, but not predetermined frequency;
a second electronic circuit, coupled to said transmit/receive antenna, having wideband receiver compatibility comprising means for optimal reception within a predetermined distance range from the object, and
a signal processor to transform the return signals into a resulting narrowband return signal having sufficient intensity to be distinguishable from ambient noise.

19. The apparatus according to claim 18, wherein the work area is a surgical site and the tag element is of such relatively small size as not to impede the functional use of an object to which it is affixed, the object being either deformable or non-deformable.

20. The apparatus according to claim 18, wherein the first and second electronic circuits are contained in a handle portion to which the transmit/receive antenna is detachably connected, the handle portion and the transmit/receive antenna constitute a hand-held scanning detection device.

21. The apparatus according to claim 20, wherein the transmit/receive antenna includes plural ring-shaped antennas for the emitting of a pulsed wideband signal as an electromagnetic signal in each coordinate direction of a multi-directional coordinate system employed.

22. The apparatus according to claim 21, wherein the antenna portion includes three mutually orthogonal ring-shaped antennas for the transmitting of the electromagnetic signal in the X, Y and Z-directions, respectively, of an X, Y and Z-coordinate system.

23. The apparatus according to claim 22, wherein the tag element is a low Q tag element.

24. The apparatus according to claim 18, wherein the tag element is a low Q tag element.

25. A method for the detection of one or more foreign objects used in a surgical site, comprising:
attachably providing each foreign object used in surgery with a much smaller tag element which does not interfere with utilization of the foreign object,
wherein the tag element includes means for responding to a wideband signal, with a response signal centered about a specific but not a predetermined frequency; and
after completion of a surgical procedure, scanning the surgical site with a scanning detection device which includes a transmitter and receiver, the transmitter emitting either one of a pulse-width modulated wideband interrogation signal or a voltage-modulated wideband interrogation signal, the wideband interrogation signal containing a frequency at which the tag element responds with a single response signal for each emitted pulse reaching the tag element, each pulse of the wideband interrogation signal being of such duration as to cause the return signals from the tag element to become cumulatively increased in intensity, resulting in a narrowband return signal having sufficient intensity to be distinguishable from background noise, to facilitate detection of the tag element and object attached thereto.

26. The method according to claim 25, wherein the scanning detection device is a handheld device including (i) a handle portion containing electrical components of the transmitter and receiver, and (ii) an antenna portion shared for both transmit and receive functions which is detachably connected to the handle portion, the antenna portion includes plural ring-shaped antennas for the emitting of a pulsed wideband signal as an electromagnetic signal in each coordinate direction of a multi-directional coordinate system.

27. The method according to claim 26, wherein the antenna portion includes three mutually orthogonal ring-shaped antennas for the transmitting of the electromagnetic signal in the X, Y and Z-directions, respectively, of an X, Y and Z-coordinate system.

28. The method according to claim 27, wherein the tag element is a low Q tag element.

29. The method according to claim 25, wherein the tag element is a low Q tag element.
